(19) United States
(12) Patent Application Publication
(10) Pub. No.: US 2015/0297385 A1
(43) Pub. Date: Oct. 22, 2015

(54) Toe Protector

(71) Applicant: Fusco Industrial Corporation, Taipei City (TW)

(72) Inventor: Chen-Yu Tsai, Taipei City (TW)

(73) Assignee: Fusco Industrial Corporation, Taipei City (TW)

(21) Appl. No.: 14/257,677

(22) Filed: Apr. 21, 2014

(51) Int. Cl. A61F 5/01

(52) U.S. Cl. CPC A61F 5/019

(57) ABSTRACT

A toe protector includes a main body made from soft and flexible materials, formed with a big-toe hole, a second toe hole and a partition block located therebetween, the main body having a first peripheral wall of U-shaped cross section extending laterally from a first side of the big-toe hole such that after extension of a big toe through the big-toe hole, the first peripheral wall encloses the big toe, the one side of the big-toe hole and parts of a foot, the main body further having a second peripheral wall of C-shaped cross section extending from a second side of the second toe hole such that after extension of a second toe through the second toe hole, the second peripheral wall encloses the second side of the second toe hole to confine the partition block between the big-toe hole and the second toe hole.

### BACKGROUND OF THE INVENTION

1. Field of the Invention

The present invention relates generally to a toe protector, and more particularly to a toe protector that is adapted to be sleeved around the toes of the human foot in order to protect the toes.

2. The Prior Arts

When a man is walking, a single foot must bear 0.8 to 1.2 times of his body weight and the reaction force generated thereby needs no emphasizing. The reaction force directly impacts the soles, the knee and the vertebrae of the man. Hence, providing a pair of insoles within the shoes is one selection in order to absorb the reaction force or impact.

When a man is walking, it is the toes and the front part of the sole that grab the ground so as to move the man body forward, only after which the feet are lifted one-by-one from the ground. It is noted that shoulders of the toes (the middle section between the distal end and the root of a toe) are bent upwardly with respect to the ground such that during the toes grabbing action against the ground, there may form clearances within a buffer pad or between the shoulders of the toes. Under this condition, the toes are not totally supported or buffered, thereby causing easy fatigue and tire to the feet in case of long walking.

In addition, owing to the general structure of a foot, the big toe and the second toe must shoulder a force greater than the remaining toes together. The lateral side of the big toe will in direct contact with the interior of the shoes such that he or she may suffer injuries, scratches in the toes or even infection upon skin rupture in case of long walking.

### SUMMARY OF THE INVENTION

A primary objective of the present invention is to provide a toe protector that is adapted to be sleeved around the toes and that is to be used within a shoe so as to protect and provides comfortable feeling to the wearer.

A toe protector of the present invention includes a main body made from soft and flexible materials, formed with a big-toe hole, a second toe hole and a partition block located therebetween, the main body having a first peripheral wall of U-shaped cross section extending laterally from a first side of the big-toe hole such that after extension of a big toe through the big-toe hole, the first peripheral wall encloses the big toe, the one side of the big-toe hole and parts of a foot, the main body further having a second peripheral wall of C-shaped cross section extending from a second side of the second toe hole such that after extension of a second toe through the second toe hole, the second peripheral wall encloses the second side of the second toe hole to confine the partition block between the big-toe hole and the second toe hole. Once a man walks after wearing the shoes with the toe protectors therein, the wearer feels comfortable owing to absorption of the reaction force by the toe protector of the present invention, thereby eliminating the problems of injuries, scratches in the toes or even infection upon skin rupture.

The materials for fabrication of the main body of the toe protector of the present invention is selected from a group consisting of TPR (Thermoplastic Rubber), TPE (Thermoplastic Elastomer), TPU (Thermoplastic polyurethanes), Silicon, Poron, PU (polyurethane), EVA, sponge, rubber. The main body of the present invention is an integral one-piece member preferably transparent or opaque and colors can be added if desired.

Another aspect of the present invention is to provide an integrally formed toe protector including a main body made from soft and flexible materials, formed with a big-toe hole, a second toe hole and a partition block located therebetween, the main body having a first peripheral wall of U-shaped cross section extending laterally from a first side of the big-toe hole such that after extension of a big toe through the big-toe hole, the first peripheral wall encloses the big toe, the one side of the big-toe hole and parts of a foot, the main body further having a second peripheral wall of C-shaped cross section extending from a second side of the second toe hole such that after extension of a second toe through the second toe hole, the second peripheral wall encloses the second side of the second toe hole to confine the partition block between the big-toe hole and the second toe hole.

The partition block located between the big-toe hole and the second toe hole provides a comfortable feeling once the toe protector is provided with the shoes. Each of the big-toe hole and the second toe hole has an axis parallel to each other. The partition block has a thickness ranging 1 mm-9 mm measured along a direction perpendicular to the axes.

Preferably, the partition block has an outwardly curved side serving as the second side of the second toe hole.

The big toe can move freely after extension through the big-toe hole, The first peripheral wall extends inclinedly with respect to the big-toe hole, a horizontal line coinciding at a point of the big-toe toe hole near the partition block cooperatively define an acute angle θ ranging 0°-50°.

Preferably, the first peripheral wall extends laterally from a first side of the big-toe hole such that after extension of a big toe through the big-toe hole, the first peripheral wall encloses the big toe, the one side of the big-toe hole and parts of a foot.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a perspective view of a toe protector of the present invention;

FIG. 2 is a front planar view of the toe protector of the present invention;

FIG. 3 is a lateral side view of the toe protector of the present invention;

FIG. 4 is a rear end view of the toe protector of the present invention;

FIG. 5 is a cross-sectional view taken along lines 5-5 in FIG. 2;

FIG. 6 is a cross-sectional view taken along lines 6-6 in FIG. 2;

FIG. 7 is a cross-sectional view taken along lines 7-7 in FIG. 2;

FIG. 8 is a cross-sectional view taken along lines 8-8 in FIG. 2;

FIG. 9 is a cross-sectional view taken along lines 9-9 in FIG. 2;

FIG. 10 is a top cross-sectional view illustrating a partition block employed in the toe protector of the present invention;

FIG. 11 is a top cross-sectional view illustrating another partition block employed in the toe protector of the present invention; and

FIG. 12 illustrates the toe protector of the present invention in application.

### DETAILED DESCRIPTION OF THE PREFERRED EMBODIMENTS

The accompanying drawings are included to provide a further understanding of the invention, and are incorporated in and constitute a part of this specification. The drawings illustrate embodiments of the invention and, together with the description, serve to explain the principles of the invention.

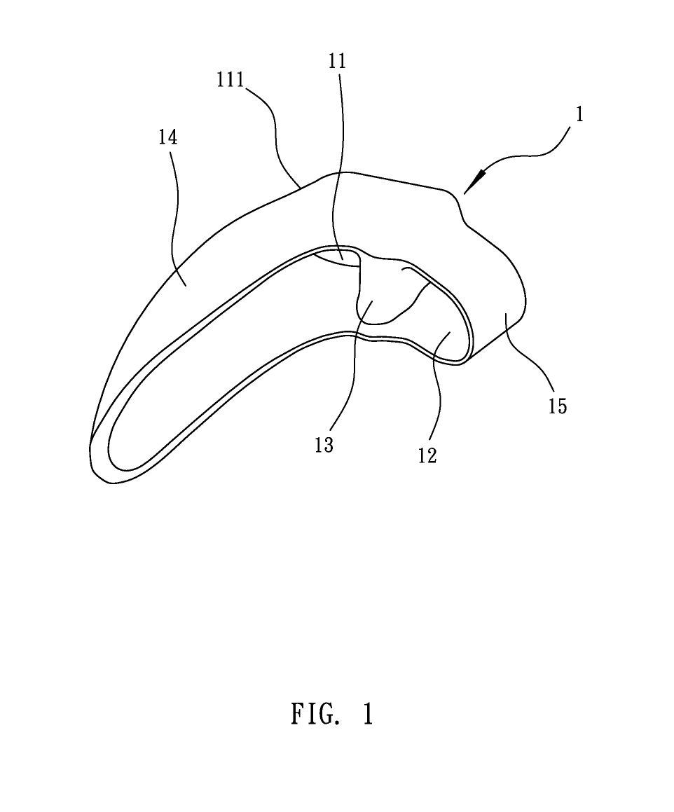

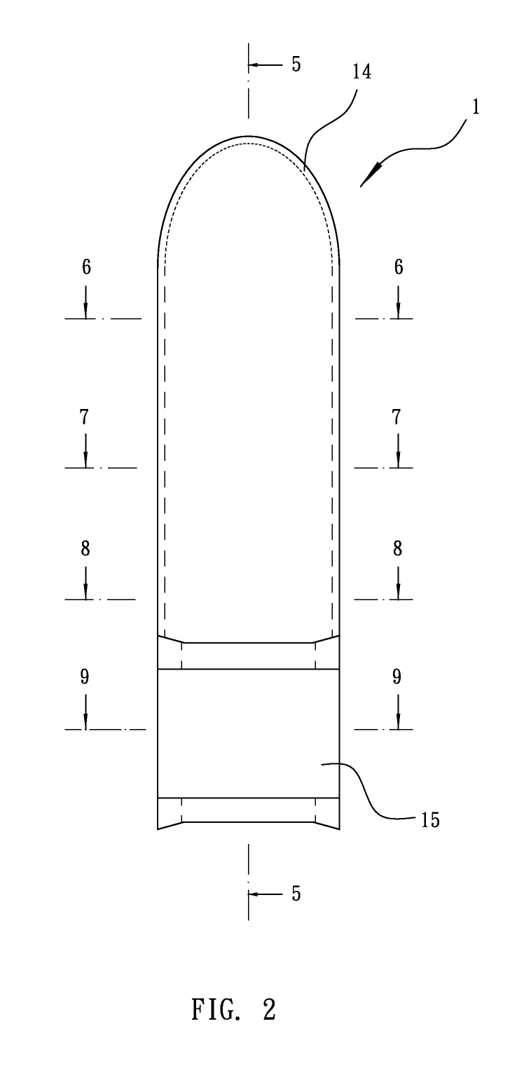

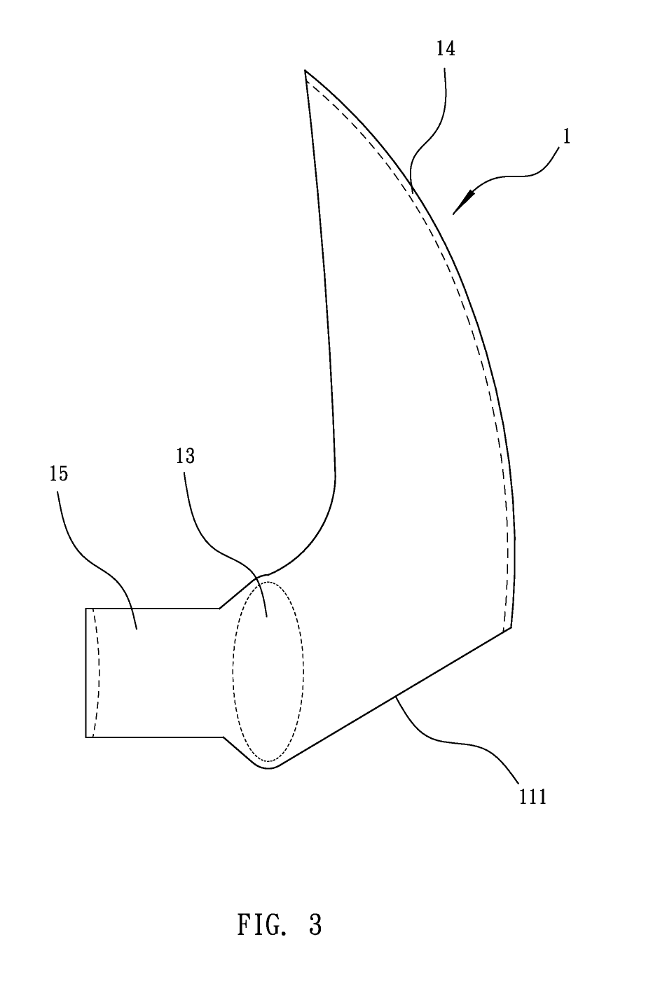

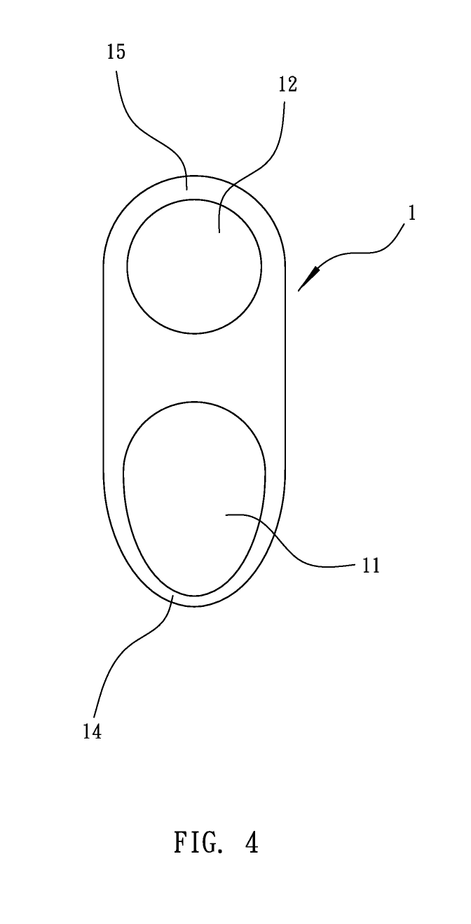

Referring to FIGS. 1 to 4, wherein FIG. 1 is a perspective view of a toe protector of the present invention; FIG. 2 is a front planar view of the toe protector of the present invention; FIG. 3 is a lateral side view of the toe protector of the present invention; and FIG. 4 is a rear end view of the toe protector of the present invention. The toe protector of the present invention includes a main body 1 made from soft and flexible materials. Preferably, the materials for fabrication of the main body of the toe protector of the present invention is selected from a group consisting of TPR (Thermoplastic Rubber), TPE (Thermoplastic Elastomer), TPU (Thermoplastic polyurethanes), Silicon, Poron, PU (polyurethane), EVA, sponge, rubber. More preferably, the main body of the present invention is an integral one-piece member that is transparent or opaque and colors can be added if desired.

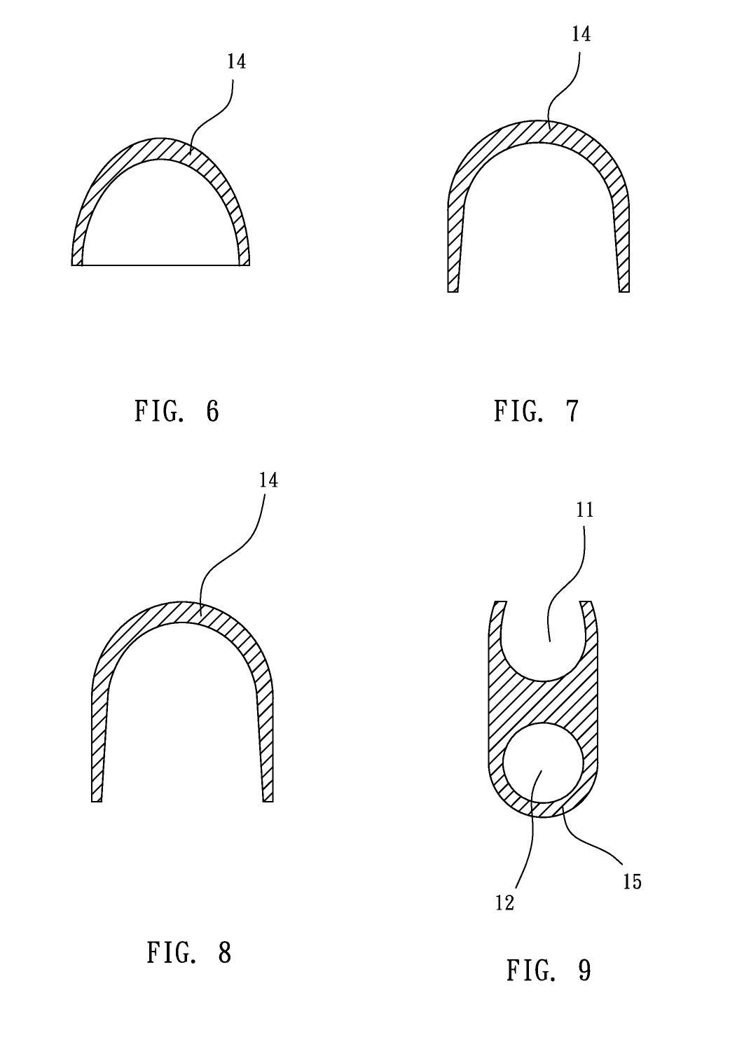

As illustrated, the main body 1 of the toe protector according to the present invention is made from soft and flexible materials selected from above stated substances. The main body 1 is formed with a big-toe hole 11, a second toe hole 12 located adjacent to said big-toe hole and a partition block 13 located between said big-toe hole 11 and said second toe hole 12. The main body 1 has a first peripheral wall 14 of U-shaped cross section extending laterally from a first side of the big-toe hole 11 in such a manner that after extension of a big toe through the big-toe hole 11, the first peripheral wall 14 is adapted to enclose the big toe, the one side of the big-toe hole 11 and parts of upper and lower of the foot, as best shown in FIG. 12. The U-shaped cross section of the first peripheral wall 14 is taken along lines 6-6, 7-7 and 8-8 in FIG. 2. In order to suit the curvature of big toe of different peoples, the depth or the curvature of the first peripheral wall 14 differs from one another, as best shown in FIGS. 6-9.

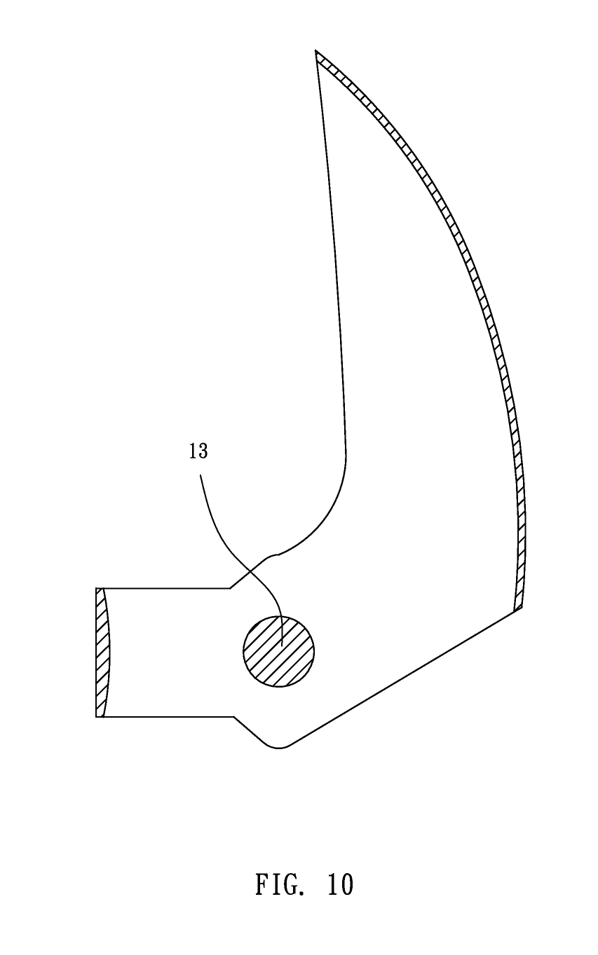

The main body 1 further has a second peripheral wall 15 of C-shaped cross section (taken along lines 9-9 in FIG. 9) extending from a second side of the second toe hole 12 in such a manner that after extension of a second toe through the second toe hole 12, the second peripheral wall 15 is adapted to enclose the second side of the second toe hole 12 so as to confine the partition block 13 between the big-toe hole 11 and the second toe hole 12.

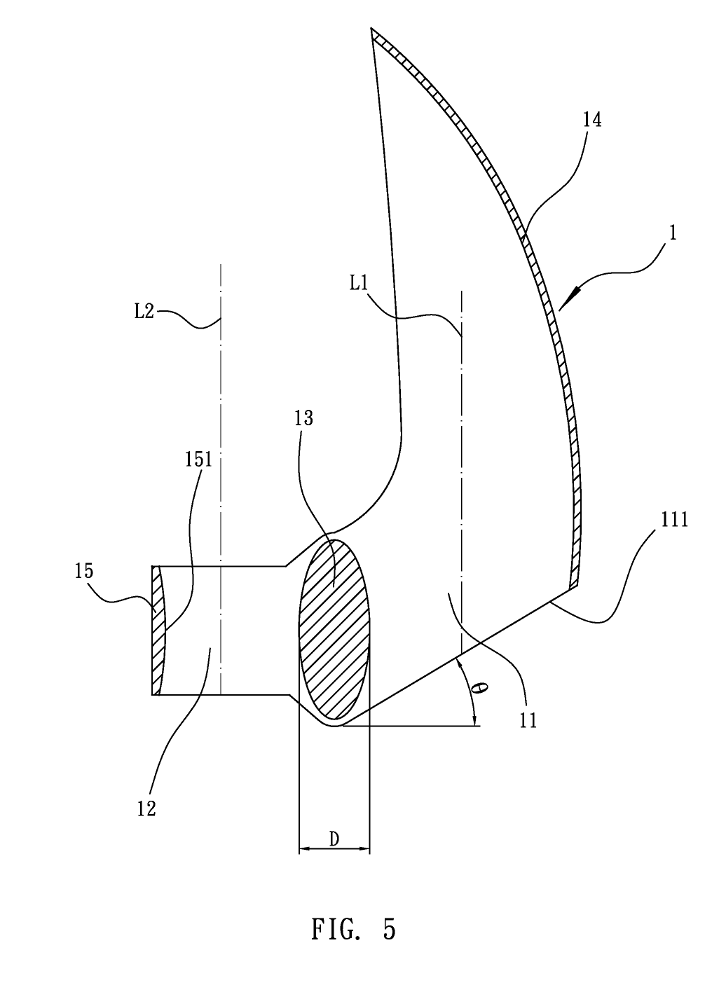

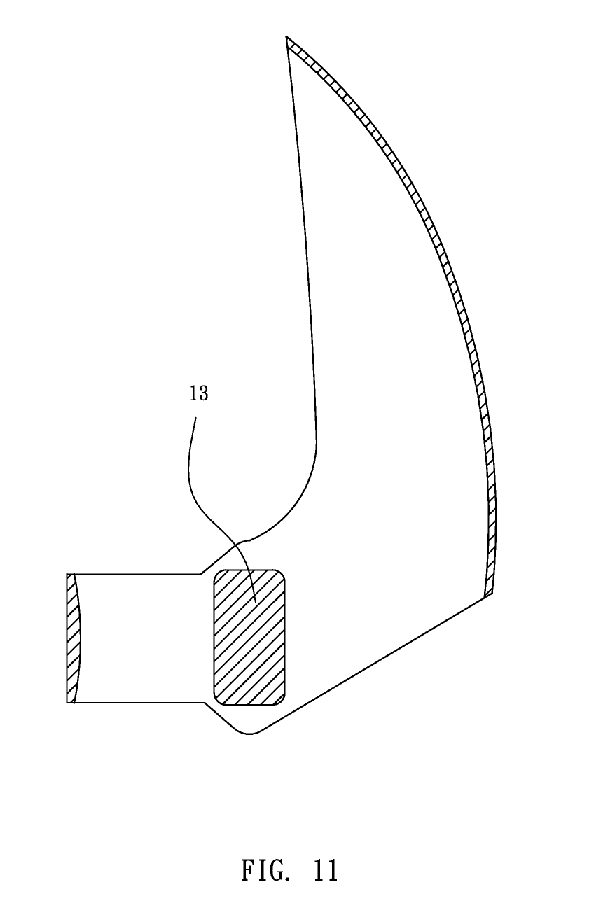

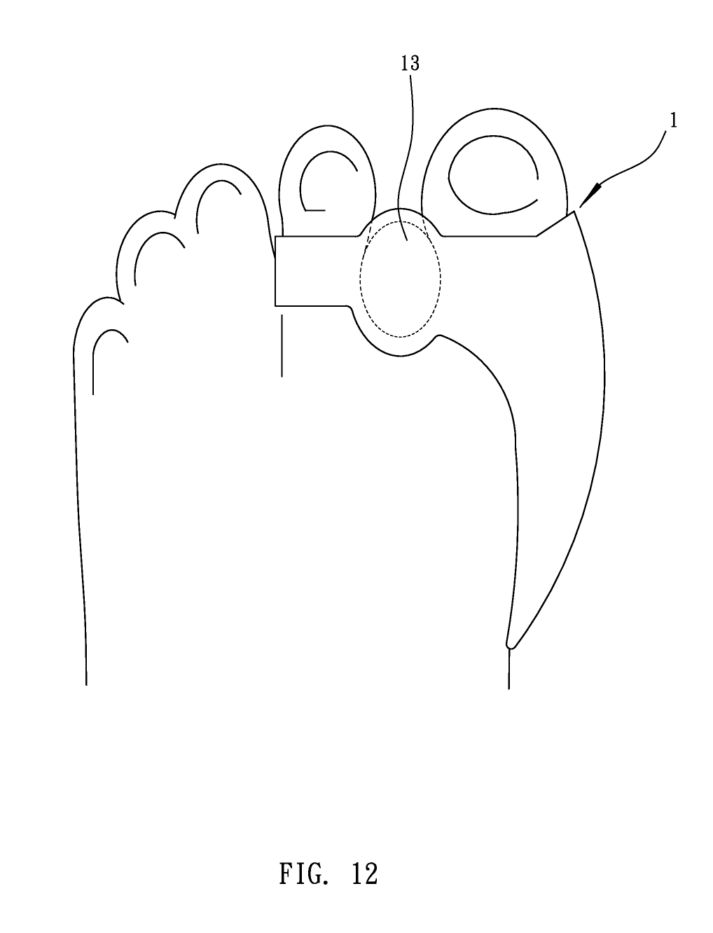

Each of the big-toe hole 11 and the second toe hole 12 has an axis L1, L2 parallel to each other. In order to provide the maximum comfort after wearing the main body r of the present invention, the partition block 13 has a thickness D ranging 1 mm-9 mm measured along a direction perpendicular to the axes L1, L2 (see FIG. 5). Preferably, the partition block 13 is oval-shaped (see FIG. 5), a circle shape (see FIG. 10) and a rectangle (see FIG. 11) or any other configurations.

As best shown in FIG. 5, the partition block 13 has an outwardly curved side serving as the second side of the second toe hole 12. In other words, the second side of the second toe hole 12 is generally curved in order to suit the curvature of an individual person.

In addition, the big toe can move freely after extension through the big-toe hole 11, The first peripheral wall 14 has a lowest portion 111 that extends inclinedly with respect to the big toe hole 11, a horizontal line coinciding at a point of the big-toe hole 11 near the partition block 13 cooperatively define an acute angle θ ranging 0°-50°.

FIG. 12 illustrates the toe protector of the present invention in application. Once a person puts on the shoes after sleeving the main body 1 of the toe protector of the present invention on the toes, he will feel comfortable when he walks or stand owing to reaction absorption by the main body 1 and due to no injuries as happened in the prior art.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A toe protector for a foot, comprising:
a main body made from soft and flexible materials and formed with a big-toe hole, a second toe hole located adjacent to said big-toe hole and a partition block located between said big-toe hole and said second toe hole, said main body having a first peripheral wall of U-shaped cross section extending laterally from a first side of said big-toe hole in such a manner that after extension of a big toe through said big-toe hole, said first peripheral wall is adapted to enclose the big toe, said one side of said big-toe hole and parts of upper and lower of the foot, said main body further having a second peripheral wall of C-shaped cross section extending from a second side of said second toe hole in such a manner that after extension of a second toe through said second toe hole, said second peripheral wall is adapted to enclose said second side of said second toe hole so as to confine said partition block between said big-toe hole and said second toe hole.

2. The toe protector according to claim 1, wherein each of said big-toe hole and said second toe hole has an axis parallel to each other, said partition block having a thickness ranging 1 mm-9 mm measured along a direction perpendicular to said axes.

3. The toe protector according to claim 2, wherein said partition block has an outwardly curved side serving as said second side of said second toe hole.

4. The toe protector according to claim 3, wherein the first peripheral wall and a horizontal line coinciding at a point of said big-toe hole near said partition block cooperatively define an acute angle θ ranging 0°-50°.

5. The toe protector according to claim 1, wherein said first peripheral wall extends curvedly and laterally from said first side of said big-toe hole.
